(19) United States
(12) Patent Application Publication
(10) Pub. No.: US 2018/0043382 A1
(43) Pub. Date: Feb. 15, 2018

(54) Handheld Aroma Diffuser

(71) Applicant: Puzhen Life Co., Limited, Hong Kong (HK)

(72) Inventor: Dong Sheng Li, Hong Kong (HK)

(21) Appl. No.: 15/271,241

(22) Filed: Sep. 21, 2016

(30) Foreign Application Priority Data
Aug. 10, 2016 (HK) 16109552.3

(51) Int. Cl. B05B 11/00; A61M 21/00; A61M 11/00

(52) U.S. Cl. CPC B05B 11/0037; A61M 11/00; A61M 21/00; A61M 2021/0016

(57) ABSTRACT

The present invention relates to a handheld aroma diffuser having a body. The body has a body housing, a protective rear casing and a battery protecting casing that are respectively connected to the body housing in a detachable manner. A placing groove and a battery groove are disposed on the body housing. An atomizing device is contained in the placing groove. A battery device is contained in the battery groove, and the battery device is electrically connected to the atomizing device. The protective rear casing covers the placing groove, and the battery protecting casing covers the battery groove. A mist discharge orifice communicated with the placing groove is disposed on the body housing and connected to the diffusion orifice of the atomizing device in an abutted manner. The atomizing device of the present invention is replaceable to lengthen the service life of the aroma diffuser and reduce use cost.

### CROSS-REFERENCE TO RELATED APPLICATIONS

The present application claims the benefit of Hong Kong Patent Application No. 16109552.3 filed on Aug. 10, 2016, the entire content of which is hereby incorporated by reference.

### TECHNICAL FIELD

The present invention relates to an aroma diffuser, particularly to a handheld aroma diffuser.

### DESCRIPTION OF THE BACKGROUND

Following the improvement of people's living standard, air conditions are widely used, so people are liable to tight skin, dry mouth and tongue, cough, common cold and other air conditioning diseases. After plant essential oil or medical liquid is added to water, the aroma diffuser splits water and the dissolved plant essential oil or medical liquid into tiny misty particles and diffuses them into the air to keep the room in high humidity and full of a fragrant smell and make the plant essential oil or medical liquid more easily absorbed by human body. The aroma diffuser plays a role in disease treatment, soul nurturing, health care and physical therapy, and particularly shows an excellent auxiliary effect on treatment of skin allergy, insomnia, common cold, cough and asthma.

At present, the atomizing devices of the existing handheld aroma diffusers are not replaceable. After the mixed liquid of water, essential oil and so on in an atomizing device is used up, the atomizing device will be discarded. It cannot be used cyclically, so the service life is short and use cost is high.

### SUMMARY OF THE INVENTION

The object of the present invention is to overcome the shortcoming of the prior art and provide an aroma diffuser with a replaceable atomizing device.

The present invention provides a handheld aroma diffuser, comprising a body. The body comprises a body housing as well as a protective rear casing and a battery protecting casing that are respectively connected to the body housing in a detachable manner; a placing groove and a battery groove are disposed on the body housing, an atomizing device is contained in the placing groove, and a battery device is contained in the battery groove and electrically connected to the atomizing device; the protective rear casing covers the placing groove, and the battery protecting casing covers the battery groove; a mist discharge orifice communicated with the placing groove is disposed on the body housing and connected to the diffusion orifice of the atomizing device in an abutted manner.

Further, the atomizing device comprises a water tank element and an atomizing element, and the water tank element is connected to the atomizing element in a detachable manner.

Further, the water tank element comprises a water tank body, a liquid storage cavity is formed inside the water tank body, and a liquid discharge orifice is disposed on one side face of the water tank body and communicated with the liquid storage cavity; there is a notch on the liquid discharge orifice; the atomizing element comprises an atomizing casing and an atomizing module mounted inside the atomizing casing; the diffusion orifice is disposed on one side face of the atomizing casing and runs through the atomizing casing and the atomizing module; a buckle is disposed at one end of the diffusion orifice, and a lug is disposed on the buckle; the buckle is snapped into the liquid discharge orifice and the lug is snapped into the notch; the other end of the diffusion orifice is connected to the mist discharge orifice in an abutted manner.

Further, the atomizing casing comprises an atomizing front casing and an atomizing rear casing connected to the atomizing front casing in a snapped manner; the atomizing module comprises a seal ring, an atomizing piece mounted to the seal ring, and a left spring piece and a right spring piece respectively connected to the anode and cathode of the atomizing piece, the seal ring is mounted to the atomizing front casing, and the left spring piece and the right spring piece are mounted to the atomizing rear casing; two spring piece openings respectively corresponding to the left spring piece and the right spring piece are disposed on the atomizing front casing.

Further, the atomizing front casing comprises a front body and two front mounting portions formed on the left and right sides of the front body, and two spring piece outlets are disposed on the front mounting portions; the seal ring is mounted to the front body; the atomizing rear casing comprises a rear body and two rear mounting portions formed on the left and right sides of the rear body, and the left spring piece and the right spring piece are respectively mounted to the two rear mounting portions.

Further, front slots are disposed on the front body, front blocks are contained in the front slots, and front hooks are formed at the terminals of the front blocks; front lugs are disposed on the two front mounting portions respectively, and front through holes are disposed on the front lugs; rear lugs are disposed on the rear body, and rear through holes are disposed on the rear lugs; the rear lugs match the front slots, and the front hooks are coupled with the rear through holes in a snapped manner; rear slots are disposed on the two rear mounting portions respectively, rear blocks are contained in the rear slots, and rear hooks are formed at the terminals of the rear blocks; the front lugs match the rear slots, and the rear hooks are coupled with the front through holes in a snapped manner.

Further, notches are disposed on the seal ring, and the front blocks match the notches.

Further, a seal ring is sleeved on the buckle, clamped between the water tank body and the atomizing casing, and received in a concave cavity on one side face of the water tank body.

Further, the water tank element further comprises a water tank cover, the upper end of the water tank body forms a liquid inlet end, and the water tank cover covers the liquid inlet end; the liquid inlet end has a liquid inlet communicated with the liquid storage cavity.

Further, a connector is disposed on the liquid inlet end, and one end of the connector is connected to the water tank cover.

Further, an O ring is disposed between the water tank cover and the liquid inlet end.

Further, the battery device comprises a battery box mounted inside the battery groove, a substrate mounted to the battery box and two electrodes; the substrate is electrically connected to the battery box, and two conductive springs are disposed on the substrate; the two electrodes are respectively mounted inside the placing groove, one ends of the two electrodes are respectively connected to the two conductive springs in an abutted manner, and the other ends pass through the two spring piece openings and are respectively connected to the left spring piece and the right spring piece in an abutted manner.

Further, a button is disposed on the substrate, and one end of the button is received in a mounting hole of the body housing.

Further, the battery box comprises a body, batteries mounted inside the body and two battery chips, and the two battery chips are respectively connected to an anode and a cathode of the batteries.

Further, the body housing comprises a protective middle casing and a protective front casing mounted to the protective middle casing; the placing groove and a battery groove are disposed on the protective middle casing; the mist discharge orifice is disposed on the protective front casing, and a connecting hole communicated with the placing groove and the mist discharge orifice is disposed on the protective middle casing; a seal ring is disposed between the mist discharge orifice and the connecting hole, and one end of the seal ring passes through the connecting hole, is located inside the placing groove and is connected to the diffusion orifice of the atomizing device in an abutted manner.

Further, buckles are respectively disposed on the protective rear casing and the battery protecting casing, catches corresponding to the buckles are disposed on the protective middle casing, and the buckles are coupled with the catches in a snapped manner.

Compared with the prior art, the present invention has the following beneficial effect: The protective rear casing and the battery protecting casing of the present invention are connected to the body housing in a detachable manner, and the atomizing device is contained in the placing groove of the body housing, so the atomizing device and the battery device can be easily repaired and replaced. After the mixed liquid of water, essential oil and so on in the atomizing device is used up, the atomizing device may be replaced or water, essential oil and so on may be added to the atomizing device again, or when the atomizing device is damaged, it may be replaced, so the aroma diffuser of the present invention may be used cyclically to lengthen service life and reduce use cost. The present invention features a simple structure and good appearance and is handy, movable, portable and suitable for popularization.

### BRIEF DESCRIPTION OF THE DRAWINGS

FIG. 1 is a front view of a handheld aroma diffuser provided in an embodiment of the present invention;

FIG. 2 is a rear view of the handheld aroma diffuser shown in FIG. 1;

FIG. 3 is an exploded view of the handheld aroma diffuser shown in FIG. 1;

FIG. 4 is a sectional view of the handheld aroma diffuser shown in FIG. 1;

FIG. 5 is an exploded view of a body housing of the handheld aroma diffuser shown in FIG. 3;

FIG. 6 is a structural schematic of the body housing shown in FIG. 5;

FIG. 7 is a structural schematic of a water tank element of the atomizing device shown in FIG. 3;

FIG. 8 is an exploded view of the water tank element shown in FIG. 7;

FIG. 9 is a structural schematic of an atomizing element of the atomizing device shown in FIG. 3;

FIG. 10 is an exploded view of the atomizing element shown in FIG. 9;

FIG. 11 is an exploded view of the battery device shown in FIG. 4.

### DETAILED DESCRIPTION OF PREFERRED EMBODIMENTS

Below the present invention will be further described by referring to the accompanying drawings and embodiments.

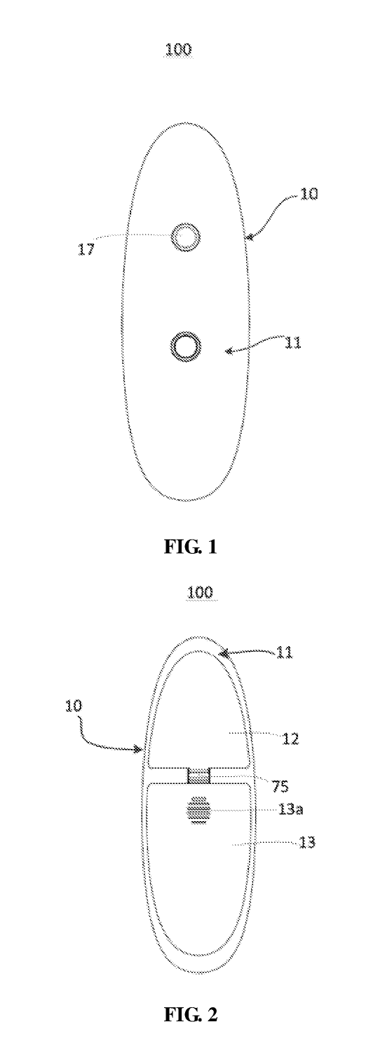

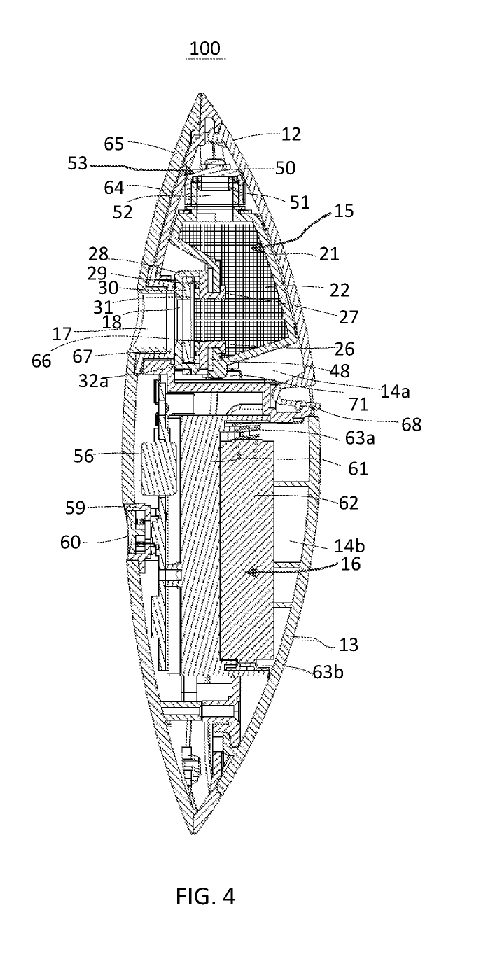

As shown in FIG. 1˜FIG. 4, the present invention provides a handheld aroma diffuser 100, comprising a body 10. The body 10 is roughly in a shuttle shape, has good appearance and is handy and portable. The body 10 comprises a body housing 11 as well as a protective rear casing 12 and a battery protecting casing 13 that are respectively connected to the body housing 11 in a detachable manner. Catches 75 are disposed at the bottom end of the protective rear casing 12. Skidproof veins 13a are disposed on the battery protecting casing 13 to make for the disassembly and assembly of the protective rear casing 12 and the battery protecting casing 13. A placing groove 14a and a battery groove 14b are disposed on the body housing 11. An atomizing device 15 is contained in the placing groove 14a. The shape of the atomizing device 15 roughly matches the shape of the placing groove 14a. The protective rear casing 12 covers the placing groove 14a and is intended to fix and protect the atomizing device 15. Therefore, the atomizing device 15 may be replaced only by removing the protective rear casing 12, thereby reducing use cost and lengthening service life of the aroma diffuser. Therefore, the aroma diffuser may be used cyclically. A mist discharge orifice 17 communicated with the placing groove 14a is further disposed on the body housing 11 and connected to the diffusion orifice 18 of the atomizing device 15 in an abutted manner. After the atomizing device 15 splits the mixed liquid of water, essential oil and so on into tiny misty particles, it pushes misty particles to the diffusion orifice 18 and the mist discharge orifice 17 and to the air to keep the room in high humidity and full of a fragrant smell and make the essential oil more easily absorbed by human body. The aroma diffuser plays a role in disease treatment, soul nurturing, health care and physical therapy. A battery device 16 is disposed inside the battery groove 14b. The battery protecting casing 13 covers the battery groove 14b and is intended to fix and protect the battery device 16. The battery device 16 is electrically connected to the atomizing device 15 and intended to supply power to the atomizing device 15.

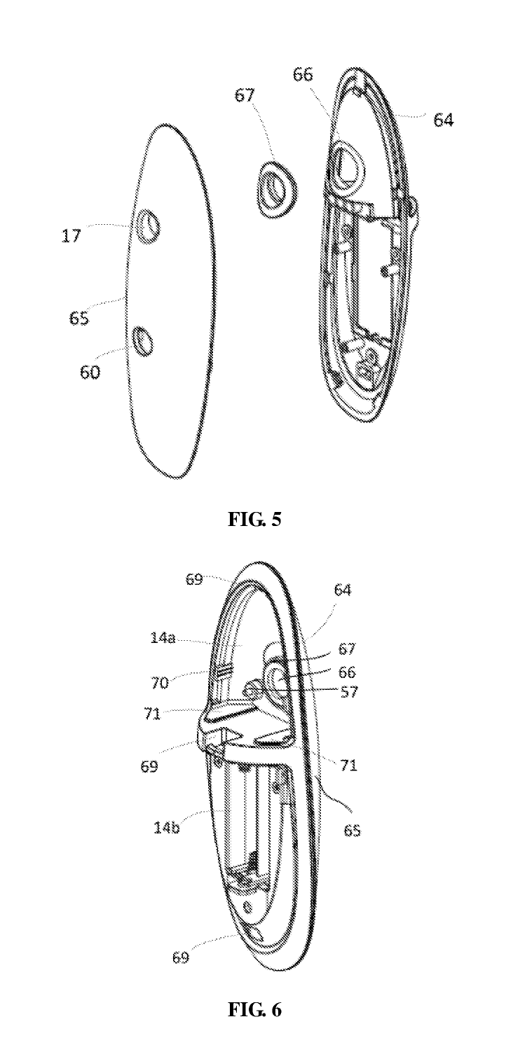

As shown in FIG. 4, FIG. 5 and FIG. 6, the body housing 11 comprises a protective middle casing 64 and a protective front casing 65 mounted to the protective middle casing 64. Specifically, the protective middle casing 64 is connected to the protective front casing 65 in a snapped manner. The foregoing placing groove 14a and battery groove 14b are disposed on the protective middle casing 64. Bosses 71 are disposed on the left and right sides of the bottom surface of the placing groove 14a and intended to place the atomizing device 15. Arrises 70 are disposed on the left and right side walls of the placing groove 14a and intended to increase friction with the atomizing device 15 and prevent shake of the atomizing device 15. The forgoing mist discharge orifice 17 is disposed on the protective front casing 65. A connecting hole 66 communicated with the placing groove 14a and the mist discharge orifice 17 respectively is disposed on the protective middle casing 64. A seal ring 67 is clamped between the mist discharge orifice 17 and the connecting hole 66 to improve sealing performance. One end of the seal ring 67 passes through the connecting hole 66 and is located inside the placing groove 14a and connected to the diffusion orifice 18 of the atomizing device 15 in an abutted manner to make for discharge of misty particles from the mist discharge orifice 17 to the air.

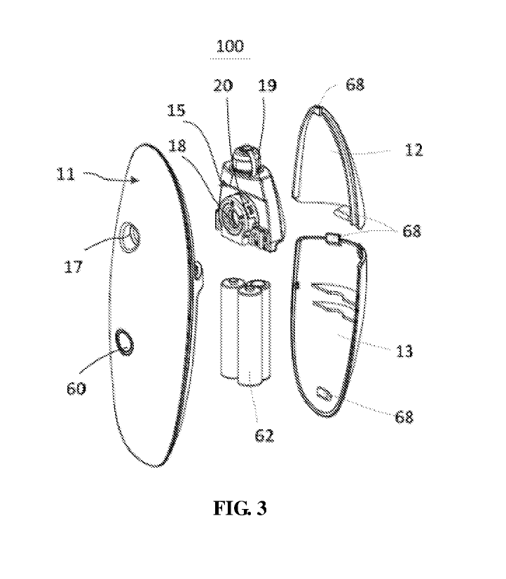

As shown in FIG. 3 and FIG. 6, buckles 68 are disposed at the upper end and bottom end of the protective rear casing 12. The buckles 68 at the bottom end are connected to the catches 75 of the foregoing protective rear casing 12 into an integral body, and their sections are in a shape similar to “<”, as shown in FIG. 4. Buckles 68 are disposed at the upper end and bottom end of the battery protecting casing 13. Catches 69 corresponding to buckles 68 are disposed on the protective middle casing 64 and coupled with the buckles 68 in a snapped manner to make for the disassembly and assembly of the protective rear casing 12 and the battery protecting casing 13, and for the repair and replacement of the atomizing device 15 and the battery device 16.

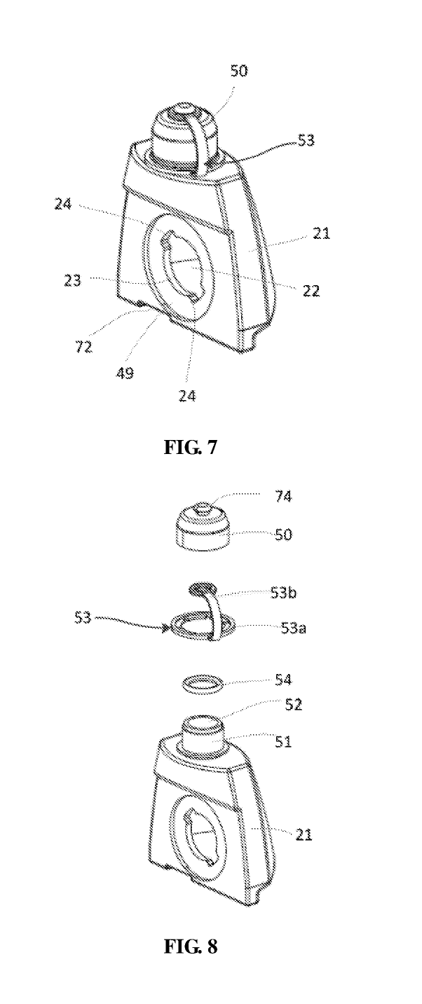

As shown in FIG. 3, FIG. 4, FIG. 7 and FIG. 8, the atomizing device 15 comprises a water tank element 19 and an atomizing element 20. The water tank element 19 comprises a water tank body 21 and a water tank cover 50. A liquid storage cavity 22 is formed inside the water tank body 21 and intended to store a mixed liquid of water, essential oil and so on. A liquid inlet end 51 is formed at the upper end of the water tank body 21. The water tank cover 50 covers the liquid inlet end 51. The liquid inlet end 51 has a liquid inlet 52 communicated with the liquid storage cavity 22. When the liquid in the liquid storage cavity 22 is used up, liquid may be added to the liquid storage cavity 22 via the liquid inlet 52 to lengthen service life. A connector 53 is disposed on the liquid inlet end 51 and comprises a connector body 53a and a connecting piece 53b formed integrally with the connector body 53a. The connector body 53a is in a ring shape and sleeved on the liquid inlet end 51. The terminal of the connecting piece 53b is in a ring shape and sleeved on a convex column 74 at the upper end of the water tank cover 50. When the water tank cover 50 is open, the connector 53 is intended to connect the water tank cover 50 so that the water tank cover 50 is not easily lost. In this embodiment, an O ring 54 is disposed between the water tank cover 50 and the liquid inlet end 51 to improve sealing performance.

A liquid discharge orifice 23 is disposed on one side face of the water tank body 21 and communicated with a liquid storage cavity 22. The liquid inside the liquid storage cavity 22 may flow out via the liquid discharge orifice 23. A notch 24 is disposed on the liquid discharge orifice 23. A concave portion 72 is disposed at the bottom end of the water tank body 21.

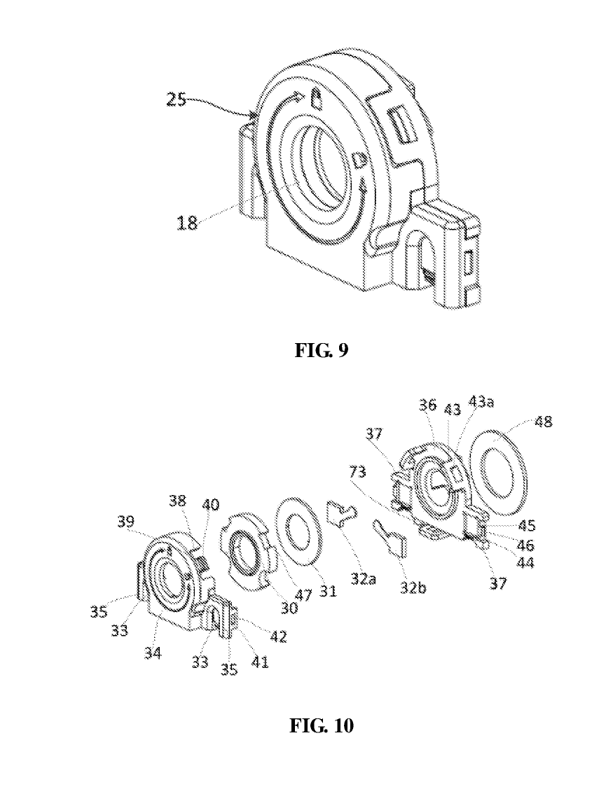

As shown in FIG. 3, FIG. 4, FIG. 9 and FIG. 10, the atomizing element 15 comprises an atomizing casing 25 and an atomizing module mounted inside the atomizing casing 25. The foregoing diffusion orifice 18 is disposed on one side face of the atomizing casing 25 and runs through the atomizing casing 25 and the atomizing module. A buckle 26 is disposed at one end of the diffusion orifice 18 and in a ring shape. A lug 27 is disposed on the buckle 26. The buckle 26 is snapped into the liquid discharge orifice 23 of the water tank body 21 so that the liquid in the liquid storage cavity 22 can enter the atomizing module via the liquid discharge orifice 23 and the diffusion orifice 18. The lug 27 is snapped into a notch 24 on the liquid discharge orifice 23. In this embodiment, a seal ring 30 is sleeved on the buckle 26 to improve sealing performance, clamped between the water tank body 21 and the atomizing casing 25 and located inside a concave cavity 49 on one side face of the water tank body 21, as shown in FIG. 7. The other end of the diffusion orifice 18 is connected to a mist discharge orifice 17 in an abutted manner.

A convex portion 73 is disposed at the bottom end of the atomizing casing 25 and matches the concave portion 72 at the bottom end of the water tank body 21. Thus, the atomizing element 20 and the water tank element 19 are detachable and both of them are replaceable. The disassembly and assembly are convenient.

The atomizing casing 25 comprises an atomizing front casing 28 and an atomizing rear casing 29. The atomizing module comprises a seal ring 30, an atomizing piece 31 mounted to the seal ring 30, and a left spring piece 32a and a right spring piece 32b connected to the anode and cathode of the atomizing piece 31. The atomizing piece 31 is intended to split liquid into tiny misty particles and push the misty particles out from a diffusion orifice 18 and a mist discharge orifice 17. The left spring piece 32a and the right spring piece 32b are intended to connect power supply so that the atomizing piece 31 can generate high-frequency vibration.

The atomizing front casing 28 comprises a front body 34 and two front mounting portions 35 formed on the left and right sides of the front body 34. The front body 34 and the two front mounting portions 35 are formed integrally or separately. The two front mounting portions 35 are mounted symmetrically. Two spring piece outlets 33 are respectively disposed on the two front mounting portions 35. Front slots 38 are disposed on the front body 34. In this embodiment, there are three front slots 38. Front blocks 39 are contained in the front slots 38. Front hooks 40 are formed at the terminals of the front blocks 39. Notches 47 are disposed on the seal ring 30. The front block 39 match the notches 47 and are intended to mount the seal ring 30. Front lugs 41 are respectively disposed on the two front mounting portions 35. Front through holes 42 are disposed on the front lugs 41.

The atomizing rear casing 29 comprises a rear body 36 and two rear mounting portions 37 formed on the left and right sides of the rear body 36. The rear body 36 and the two rear mounting portions 37 are formed integrally or separately. A left spring piece 32a and a right spring piece 32b are mounted to two rear mounting portions 37 respectively. Two spring piece outlets 33 correspond to the left spring piece 32a and the right spring piece 32b respectively. Rear lugs 43 are disposed on the rear body 36. Rear through holes 43a are disposed on the rear lugs 43. The rear lugs 43 match the front slots 38. Front hooks 40 are coupled with the rear through holes 43a in a snapped manner. Rear slots 44 are respectively disposed on the two rear mounting portions 37. Rear blocks 45 are contained in the rear slots 44. Rear hooks 46 are formed at the terminals of the rear blocks 45. Front lugs 41 match the rear slots 44. Rear hooks 46 are coupled with front through holes 42 in a snapped manner. Thus, the atomizing front casing 28 and the atomizing rear casing 29 are detachable to make for repair and replacement of the atomizing module and lengthen service life.

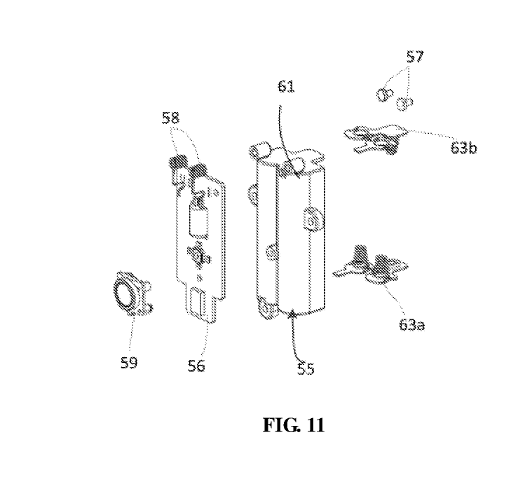

As shown in FIG. 4 and FIG. 11, the battery device 16 comprises a battery box 55 mounted inside a battery groove 14b, a substrate 56 mounted to one side face of the battery box 55, and two electrodes 57. The substrate 56 is electrically connected to the battery box 55. A button 59 and two conductive springs 58 are disposed on the substrate 56. One end of the button 59 is contained in a mounting hole 60 of the body housing 11. The two electrodes 57 are respectively mounted inside a placing groove 14a. As shown in FIG. 6, one ends of the two electrodes 57 are respectively connected to the two conductive springs 58 in an abutted manner and the other ends run through two spring piece openings 33 and are connected to the left spring piece 32a and the right spring piece 32b in an abutted manner. Through the abutted connection between the two conductive springs 58 and the two electrodes 57, and between the two electrodes 57 and the left spring piece 32a and right spring piece 32b, a channel is formed between the substrate 56 and the seal ring 31 so that the high frequency current generated by the substrate 56 can be transferred to the atomizing piece 31 and the atomizing piece 31 generates high frequency vibration.

In this embodiment, the battery box 55 comprises a body 61, batteries 62 inside the body 61, and two battery chips 63a and 63b. The two battery chips 63a and 63b are respectively connected to an anode and a cathode of the batteries 62. In this embodiment, the batteries 62 are three 3A batteries and replaceable, as shown in FIG. 3. Through the two battery chips 63a and 63b, the three batteries 62 realize series connection and power supply to the substrate 56.

Working principle of the present invention: When the button 59 is pressed, the battery 62 will supply power to the substrate 56. As the two conductive springs 58 are connected to the two electrodes 57, and the two electrodes 57 are connected to the left spring piece 32a and the right spring piece 32b, the high frequency current generated by the substrate 56 is transferred to the atomizing piece 31. The atomizing piece 31 then generates high frequency vibration. On the one hand, the high frequency vibration of the atomizing piece 31 pats the contacted liquid flowing into the diffusion orifice 18 from the water tank body 21 and splits it into tiny misty particles. On the other hand, the high frequency vibration of the atomizing piece 31 will generate certain push on its surface, thereby pushing the generated misty particles to the diffusion orifice 18 and the connecting hole 66, eventually pushing it out of the mist discharge orifice 17 into the air and achieving the function of fumigating and moisturizing.

The foregoing embodiments only represent the preferred embodiments of the present invention. Their descriptions are concrete and detailed, but they shall not be therefore understood as limitations to the scope of the present invention patent. It shall be noted that for those skilled in the art, various changes and modifications may be made to the embodiments without departing from the spirit of the present invention, such as: combinations of different features of the embodiments. All these shall be in the protective scope of the present invention.

1. A handheld aroma diffuser, comprising a body, wherein the body comprises a body housing as well as a protective rear casing and a battery protecting casing that are respectively connected to the body housing in a detachable manner; a placing groove and a battery groove being disposed on the body housing, an atomizing device being contained in the placing groove, and a battery device being contained in the battery groove and electrically connected to the atomizing device; the protective rear casing covering the placing groove, and the battery protecting casing covering the battery groove; a mist discharge orifice communicated with the placing groove being disposed on the body housing and connected to a diffusion orifice of the atomizing device in an abutted manner;
wherein the atomizing device comprises a water tank element and an atomizing element; the water tank element being connected to the atomizing element in a detachable manner;
wherein the water tank element comprises a water tank body; a liquid storage cavity being formed inside the water tank body, and a liquid discharge orifice being disposed on one side face of the water tank body and communicated with the liquid storage cavity; there being a notch on the liquid discharge orifice; the atomizing element comprising an atomizing casing and an atomizing module mounted inside the atomizing casing; the diffusion orifice being disposed on one side face of the atomizing casing and running through the atomizing casing and the atomizing module; a buckle being disposed at one end of the diffusion orifice and in a ring shape, and a lug being disposed on the buckle; the buckle being snapped into the liquid discharge orifice and the lug being snapped into the notch; the other end of the diffusion orifice being connected to the mist discharge orifice in an abutted manner; bottom end of the atomizing casing comprising a convex portion, bottom end of the water tank body comprising a concave portion, the convex portion matching the concave portion, thereby the atomizing element and the water tank element being detachable.

2. (canceled)

3. (canceled)

4. The handheld aroma diffuser according to claim 1, wherein the atomizing casing comprises an atomizing front casing and an atomizing rear casing connected to the atomizing front casing in a snapped manner; the atomizing module comprising a seal ring, an atomizing piece mounted to the seal ring, and a left spring piece and a right spring piece respectively connected to the anode and cathode of the atomizing piece, the seal ring being mounted to the atomizing front casing, and the left spring piece and the right spring piece being mounted to the atomizing rear casing; two spring piece openings respectively corresponding to the left spring piece and the right spring piece being disposed on the atomizing front casing.

5. The handheld aroma diffuser according to claim 4, wherein the atomizing front casing comprises a front body and two front mounting portions formed on left and right sides of the front body; the two spring piece outlets being disposed on the front mounting portions; the seal ring being mounted to the front body; the atomizing rear casing comprising a rear body and two rear mounting portions formed on the left and right sides of the rear body; the left spring piece and the right spring piece being respectively mounted to the two rear mounting portions.

6. The handheld aroma diffuser according to claim 5, wherein front slots are disposed on the front body; front blocks being contained in the front slots, and front hooks being formed at the terminals of the front blocks; front lugs being disposed on the two front mounting portions respectively, and front through holes being disposed on the front lugs; rear lugs being disposed on the rear body, and rear through holes being disposed on the rear lugs; the rear lugs matching the front slots, and the front hooks being coupled with the rear through holes in a snapped manner; rear slots being disposed on the two rear mounting portions respectively, rear blocks being contained in the rear slots, and rear hooks being formed at the terminals of the rear blocks; the front lugs matching the rear slots, and the rear hooks being coupled with the front through holes in a snapped manner.

7. The handheld aroma diffuser according to claim 6, wherein notches are disposed on the seal ring; the front blocks matching the notches.

8. The handheld aroma diffuser according to claim 1, wherein a seal ring is sleeved on the buckle, clamped between the water tank body and the atomizing casing, and received in a concave cavity on one side face of the water tank body.

9. The handheld aroma diffuser according to claim 1, wherein the water tank element further comprises a water tank cover; the upper end of the water tank body forming a liquid inlet end, and the water tank cover covering the liquid inlet end; the liquid inlet end having a liquid inlet communicated with the liquid storage cavity.

10. The handheld aroma diffuser according to claim 9, wherein a connector is disposed on the liquid inlet end; one end of the connector being connected to the water tank cover.

11. The handheld aroma diffuser according to claim 9, wherein an O ring is disposed between the water tank cover and the liquid inlet end.

12. The handheld aroma diffuser according to claim 4, wherein the battery device comprises a battery box mounted inside the battery groove, a substrate mounted to the battery box and two electrodes; the substrate being electrically connected to the battery box, and two conductive springs being disposed on the substrate; the two electrodes being respectively mounted inside the placing groove, one ends of the two electrodes being respectively connected to the two conductive springs in an abutted manner, and the other ends passing through the two spring piece openings and being respectively connected to the left spring piece and the right spring piece in an abutted manner.

13. The handheld aroma diffuser according to claim 12, wherein a button is disposed on the substrate; one end of the button being received in a mounting hole of the body housing.

14. The handheld aroma diffuser according to claim 12, wherein the battery box comprises a body, batteries mounted inside the body and two battery chips; the two battery chips being respectively connected to an anode and a cathode of the batteries.

15. The handheld aroma diffuser according to claim 1, wherein the body housing comprises a protective middle casing and a protective front casing mounted to the protective middle casing; the placing groove and the battery groove being disposed on the protective middle casing; the mist discharge orifice being disposed on the protective front casing, and a connecting hole communicated with the placing groove and the mist discharge orifice being disposed on the protective middle casing; a seal ring being clamped between the mist discharge orifice and the connecting hole, and one end of the seal ring passing through the connecting hole, being located inside the placing groove and being connected to the diffusion orifice of the atomizing device in an abutted manner.

16. The handheld aroma diffuser according to claim 15, wherein buckles are respectively disposed on the protective rear casing and the battery protecting casing; catches corresponding to the buckles being disposed on the protective middle casing, and the buckles being coupled with the catches in a snapped manner.
